(19) United States
(12) Patent Application Publication
(10) Pub. No.: US 2011/0245574 A1
(43) Pub. Date: Oct. 6, 2011

(54) Catalytic hydrogenation process

(76) Inventors: Qi Sun, West Chester, PA (US); Mark M. Buler, Eagleville, PA (US)

(21) Appl. No.: 12/798,195

(22) Filed: Mar. 31, 2010

(51) Int. Cl. C07C 7/00

(52) U.S. Cl. 585/841

(57) ABSTRACT

A process for the selective catalytic hydrogenation of alkynes and/or dienes in a hydrocarbon stream in the presence of hydrogen, an alcohol, and a supported catalyst is disclosed. The presence of the alcohol reduces the catalyst deactivation and improves the selectivity of the hydrogenation.

### FIELD OF THE INVENTION

The invention relates to a selective hydrogenation of alkynes and/or dienes in a hydrocarbon stream. In particular, alkynes and/or dienes are hydrogenated in the presence of a supported catalyst.

### BACKGROUND OF THE INVENTION

Steam cracking, which is the thermal cracking of hydrocarbons in the presence of steam, is used commercially to produce ethylene, propylene, butenes, butadiene, and isoprene, among others. The reaction mixture of a steam cracking process, commonly called a pyrolysis gas, is typically separated into various product is streams, including hydrogen, methane, ethylene, propylene, and butenes.

Olefins (e.g., ethylene, propylene) obtained from a steam cracking process often contain other unsaturated impurities. For example, ethylene often contains a small amount of acetylene, which can deactivate polymerization catalysts. Similarly, propylene often contains impurities such as methyl acetylene and propadiene. The impurities are generally removed by hydrogenation processes in a gas phase in the presence of solid hydrogenation catalysts. For example, U.S. Pat. No. 6,127,310 teaches the selective hydrogenation of acetylene contained in an ethylene stream produced by steam cracking in the presence of a supported palladium catalyst in the gas phase.

During the catalytic hydrogenation of a stream containing impurities such as alkynes or dienes, the catalyst tends to become deactivated due to the deposition of hydrocarbon oligomers (often called “green oil”) formed during the hydrogenation. Green oil is formed by side reactions of the hydrogenation of acetylenes or dienes. It can occur through the dimerization of acetylene to butadiene followed by oligomerization with successive addition of acetylene to a chain of molecules adsorbed on the catalyst surface. The green oil is a mixture of varying composition with a boiling point from about 120 to about 400° C. The green oil tends to stay adsorbed on the catalyst causing eventual loss of the catalyst activity. Thus it is necessary to regenerate the catalyst frequently.

Catalytic hydrogenation of alkyne or diene impurities of an olefin stream can also be performed in the presence of a liquid solvent. For example, U.S. Pat. No. 4,571,442 teaches a process for the selective hydrogenation of acetylene in a mixture of acetylene and ethylene, wherein the mixture is passed through a palladium-on-alumina catalyst in the presence of a liquid phase comprising at least one hydrocarbon and an amine compound. U.S. Pat. No. 4,587,369 teaches a similar process for the hydrogenation of a C₄ stream using an amine-containing solvent.

U.S. Pat. No. 7,288,686 teaches a process for the selective hydrogenation of a feed containing acetylenes, dienes, and olefins in at least partial liquid phase with hydrogen in the presence of a palladium catalyst for the selective hydrogenation of the acetylenes and/or dienes. A solvent such as benzene, toluene, or tetrahydrofuran may be used.

U.S. Pat. No. 7,408,091 teaches a process for the selective hydrogenation of acetylene and acetylenic compounds wherein the acetylene and/or acetylenic compounds are absorbed from a gas or liquid stream by use of a non-hydrocarbon absorbent liquid to provide a reactant stream. The reactant stream comprising the absorbent liquid containing the acetylene or acetylenic compounds is then reacted with hydrogen in the presence of a catalyst comprising a Group VIII metal. Suitable absorbents include n-methyl-2-pyrrolidone, acetone, tetrahydrofuran, dimethylsulfoxide, and monomethylamine.

Despite these efforts, catalyst deactivation continues to be a problem in converting low levels of alkynes or dienes in a mono-olefin. It is desirable to develop economic processes that give high selectivities in converting unsaturated impurities such as alkynes and/or dienes to the corresponding mono-olefins and with slow catalyst deactivation.

### SUMMARY OF THE INVENTION

The invention relates to a process for selective catalytic hydrogenation of alkynes and/or dienes in a hydrocarbon stream in the presence of hydrogen, an alcohol, and a supported catalyst. The presence of alcohol reduces the catalyst deactivation and improves the selectivity of the hydrogenation.

### BRIEF DESCRIPTION OF THE DRAWINGS

FIG. 1 shows the average temperature of the catalyst bed as a function of the reaction time in a catalytic hydrogenation of acetylene in ethylene.

FIG. 2 shows the selectivity to ethylene from the hydrogenation of acetylene as a function of the reaction time.

FIG. 3 shows the amount of ethane formed in the hydrogenation of ethylene as a function of the reaction time.

### DETAILED DESCRIPTION OF THE INVENTION

The present invention is a process comprising treating a hydrocarbon stream comprising an olefin, an alkyne and/or a diene in the presence of hydrogen, an alcohol, and a supported Group VIII metal catalyst. Suitable olefins include any hydrocarbons containing a carbon-carbon double bond. Preferably the olefin consists of carbon and hydrogen atoms only, more preferably it contains 2 to 5 carbon atoms. Examples of suitable olefins include ethylene, propylene, 1-butene, 2-butenes, and isobutene. Examples of alkynes and/or dienes include acetylene, methyl acetylene, propadiene, butadienes, ethyl acetylene, dimethyl acetylene, vinyl acetylene, pentadienes, propyl acetylenes, and the like. Generally the total amount of the alkynes and/or dienes in the hydrocarbon stream is 0.001 to 5 wt %, preferably 0.005 to 3 wt %, more preferably 0.01 to 2 wt %, and most preferably 0.02 to 1 wt %.

In one example, the hydrocarbon stream comprises propylene, propadiene, and methyl acetylene, wherein the amount of propylene is 90 to 99 wt % and the amount of propadiene and methyl acetylene combined is in the range of 0.005 to 3 wt %, preferably 0.01 to 2 wt %, more preferably 0.02 to 1 wt %. In one another example, the hydrocarbon stream comprises ethylene and acetylene, wherein the amount of ethylene is from 90 to 99 wt % and the amount of acetylene is 0.005 to 3 wt %, preferably 0.01 to 2 wt %, more preferably 0.02 to 1 wt %. In yet another example, the hydrocarbon stream comprises a butene (1-butene, 2-butenes, isobutene, or a mixture thereof) and butadiene, wherein the amount of the butene is 90 to 99 wt % and the amount of butadiene is in the range of 0.005 to 3 wt %, preferably 0.01 to 2 wt %, more preferably 0.02 to 1 wt %.

The hydrocarbon stream may comprise other components, particularly paraffins, for example, methane, ethane, propane, butanes, and the like.

The process is performed in the presence of hydrogen. The molar ratio of hydrogen to the alkynes and/or dienes is generally in the range of from 1:1 to 50:1, preferably 1:1 to 10:1. Hydrogen gas may be mixed with a hydrocarbon stream before the treatment. Alternatively, hydrogen may be fed to a reactor separately.

The process is conducted in the presence of a supported Group VIII metal catalyst. The catalyst comprises a carrier. Typically, the carrier is porous and has a surface area of from 1 to 1000 m²/g, preferably from 2 to 500 m²/g, more preferably from 5 to 200 m²/g. The carrier should be relatively refractory to the conditions utilized in the chemical process. Suitable carriers are: (1) inorganic oxides and mixed oxides such as alumina, silica or silica gel, titanium dioxide, zirconium dioxide, chromium oxide, zinc oxide, magnesia, boria, silica-alumina, silica-magnesia, alumina-boria, silica-zirconia, clays, diatomaceous earth, fuller's earth, kaolin, etc.; (2) ceramics, porcelain, crushed firebrick, and bauxite; (3) zeolites such as naturally occurring or synthetically prepared zeolites either in the hydrogen form or in a form is which has been treated with cations; and (4) combinations of members from these groups. Aluminas are preferred carriers.

The catalyst comprises a Group VIII metal. Preferred Group VIII metals are Pd, Pt, and mixtures thereof. Typically the amount of Group VIII metal present in the catalyst is in the range of from 0.005 to 20 wt %, preferably 0.01 to 5 wt %. Catalysts comprising palladium are preferred. Catalysts comprising 0.02 to 2 wt % palladium are particularly preferred.

The catalyst may further comprise a Group IB metal, Group IIB metal, or a mixture of both. Preferred metals are Ag, Au, and mixtures thereof. Ag is particularly preferred. Typically, the amount of the Group IB or Group IIB metal present in the catalyst is in the range of from 0.01 to 20 wt %, preferably 0.1 to 5 wt %.

The process is conducted in the presence of an alcohol. The alcohol can be represented by formula R—OH, wherein R is an alkyl group consisting of carbon and hydrogen only. C₁₋₅ alcohols are preferred. Suitable alcohols include methanol, ethanol, 1-propanol, 2-propanol, 1-butanol, tert-butanol, and the like, and mixtures thereof. Ethanol is particularly preferred when the hydrocarbon stream comprises ethylene and acetylene. Similarly, 1-propanol or 2-propanol is preferred when the hydrocarbon stream comprises propylene. The weight ratio of the alcohol to the hydrocarbon stream is typically in the range of 1:10 to 10:1.

The process is preferably performed under such temperatures and pressures so that at least a portion of the alcohol is in the liquid phase. Generally it is carried out at 50 to 200° C., preferably at 60 to 150° C. The pressure is generally controlled at 50 to 2000 psig, preferably at 100 to 1500 psig. Generally the temperature of the bed is increased to maintain a desired conversion of the alkynes and/or the dienes.

The process is typically conducted at a gas hourly space velocity of 100 to 10,000 h⁻¹, measured by the flow rate of the hydrocarbon stream fed to the reactor relative to the volume of the catalyst bed. Preferably it ranges from 500 to 5,000 h⁻¹.

The process is performed via any of the known reactor systems within the discretion of one skilled in the art, including trickle bed reactors, fluidized beds, fixed, or moving bed reactors, riser reactors, or any other reaction system. Preferably the process is carried out in a fixed-bed reactor.

The process may be conducted in the presence of carbon monoxide. Carbon monoxide can promote the selective hydrogenation of alkynes or dienes to their corresponding mono-olefins (U.S. Pat. No. 7,408,091). Carbon monoxide may be contained in the hydrocarbon stream, or may be fed simultaneously with the hydrocarbon stream, hydrogen, or with the alcohol. The molar ratio of carbon monoxide to hydrogen is generally 1:1 to 1:1000.

The effluent of the reaction can be separated by flashing or distillation to recover the alcohol. The heavy hydrocarbon components (e.g., green oil) present in the recovered alcohol may be isolated by distillation before the alcohol is reused in the process.

The process produces a purified hydrocarbon stream with reduced concentration of alkynes and/or dienes. Preferably the combined concentration of alkynes and dienes in the purified stream is less than 10 ppm, more preferably less than 1 ppm.

### Example 1

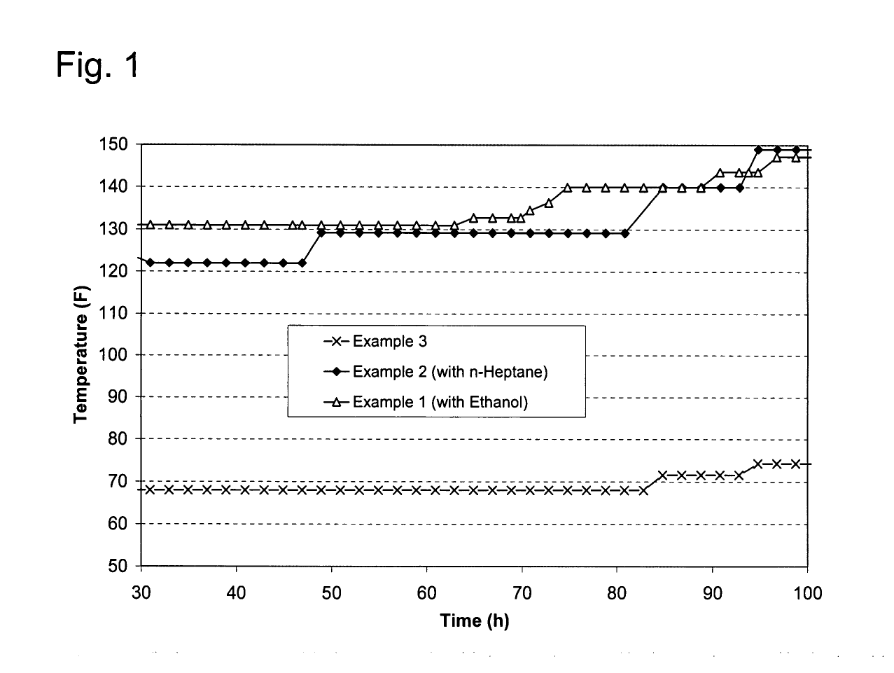

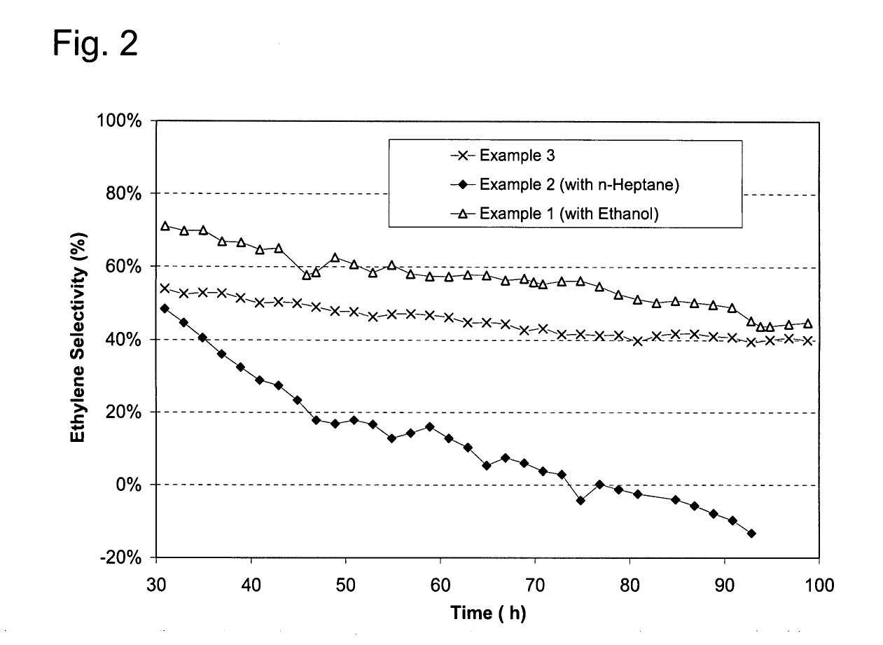

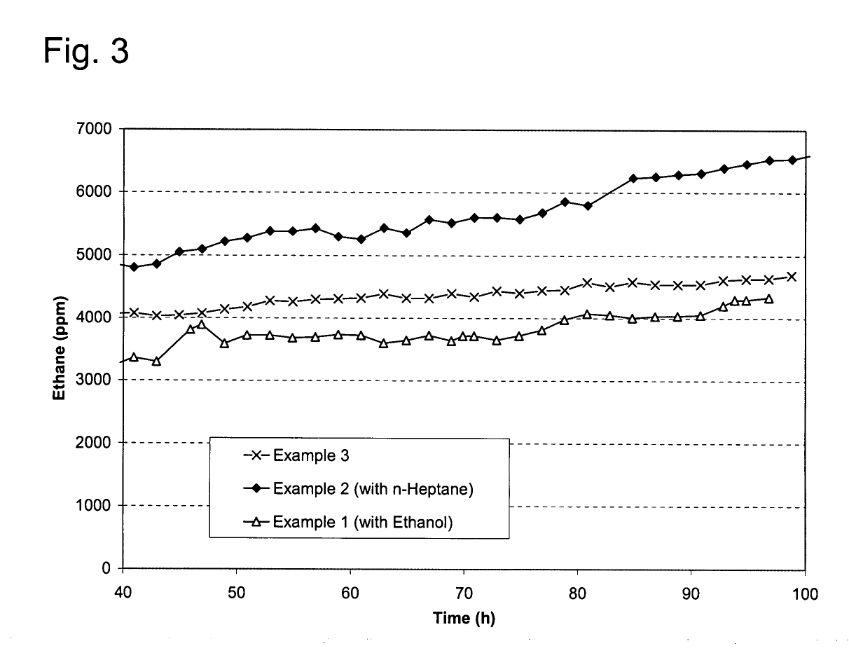

A spherical catalyst containing 0.03 wt % Pd and 0.18 wt % Ag supported on alumina (average particle diameter 4.0 mm, surface area 150 m²/g, 15 mL) is charged into a tubular reactor (ID 0.75 inch). A gas mixture containing ethylene, 1 mol % acetylene, and 1.3 mol % hydrogen is fed to the reactor. The pressure is controlled at 300 psig. The gas hourly space velocity is at 3000 h⁻¹. Ethanol is fed to the reactor at a flow rate of 0.74 mL/min. The weight ratio of ethanol to ethylene is about 1:1. The reaction temperature is adjusted so the acetylene conversion is maintained at about 70%. The average temperature of the bed as a function of the reaction time is shown in FIG. 1. The selectivity from acetylene to ethylene is shown in FIG. 2. The concentration of ethane in the reactor effluent is shown in FIG. 3.

### Comparative Example 2

The procedure of Example 1 is repeated except that n-heptane is fed to the reactor at a flow rate of 0.74 mL/min instead of ethanol.

### Comparative Example 3

The procedure of Example 1 is repeated except that ethanol is not fed to the reactor.

FIG. 1 shows the required temperature of the catalyst bed in order to maintain 70% acetylene conversion. When n-heptane or ethanol is fed to the reactor, it requires significantly higher temperatures to maintain 70% acetylene conversion as compared to Example 3. This may be because n-heptane or ethanol occupies part of the catalyst surface and thus lowers its activity. In addition, the initial catalyst bed temperature is higher in Example 1 (using ethanol) than in Example 2 (using heptane), indicating that ethanol has a greater effect on the catalyst activity than n-heptane when the catalyst is fresh. However, FIG. 1 shows that the catalyst deactivates faster in Example 2 (using n-heptane) than in Example 1 (using ethanol), as shown by the greater temperature increase necessary to maintain 70% conversion.

FIG. 2 indicates that the hydrogenation of acetylene in ethylene in the presence of n-heptane decreases the selectivity to ethylene at the same acetylene conversion and the same gas flow rate (comparing Examples 2 and 3). The presence of ethanol, however, improves the selectivity from acetylene to ethylene. The overall negative ethylene selectivity observed in Example 2 (FIG. 2) at the reaction time between 75 h and 95 h is because the amount of ethylene consumed by reaction of ethylene with hydrogen is greater than the amount of ethylene formed from the hydrogenation of acetylene.

The amount of ethane measured in the reactor effluent is shown in FIG. 3. Comparison of Examples 1 and 3 shows that the addition of ethanol to the reactor reduces the amount of ethane formed. However, the use of n-heptane increases the ethane formation, which is undesirable.

We claim:

1. A process comprising treating a hydrocarbon stream comprising an olefin and 0.001 to 5 wt % of an alkyne, a diene, or a mixture of both in the presence of hydrogen, an alcohol and a supported Group VIII metal catalyst to produce a purified hydrocarbon stream.

2. The process of claim 1 wherein at least a portion of the alcohol is in liquid phase.

3. The process of claim 1 wherein the weight ratio of the alcohol to the hydrocarbon stream is in the range of 1:10 to 10:1.

4. The process of claim 1 wherein the alcohol is a C₁₋₅ alcohol.

5. The process of claim 1 wherein the olefin is ethylene and the alcohol is ethanol.

6. The process of claim 1 wherein the olefin is propylene and the alcohol is a propanol.

7. The process of claim 1 wherein the Group VIII metal is palladium.

8. The process of claim 1 wherein the catalyst further comprises a Group IB metal, a Group IIB metal, or a mixture of both.

9. The process of claim 1 wherein the catalyst further comprises silver.

10. The process of claim 1 wherein the olefin has a concentration of from 50 to 99 wt % in the hydrocarbon stream.

11. The process of claim 1 wherein the olefin has a concentration of from 90 to 99 wt % in the hydrocarbon stream.

12. The process of claim 1 wherein the olefin is ethylene and the alkyne is acetylene.

13. The process of claim 1 wherein the catalyst comprises palladium, silver, and alumina.
